(19) United States
(12) Patent Application Publication
(10) Pub. No.: US 2003/0207275 A1
(43) Pub. Date: Nov. 6, 2003

(54) Method for detection of SP-A2 gene variants useful for prediction of predisposition to aspergillosis

(76) Inventors: Puranam Usha Sarma, Delhi (IN); Taruna Madan, Delhi (IN); Shweta Saxena, Delhi (IN)

Correspondence Address: SMITH, GAMBRELL & RUSSELL, LLP, 1850 M STREET, N.W., SUITE 800, WASHINGTON, DC, 20036, US

(21) Appl. No.: 10/102,731

(22) Filed: Mar. 22, 2002

(51) Int. Cl. C12Q 1/68; C12P 19/34

(52) U.S. Cl. 435/6; 435/91.2

(57) ABSTRACT

The present invention relates to allelic variants of human SP-A2 gene and provides allele specific primers and probes suitable for detecting these allelic variants for applications such as molecular diagnosis, prediction of an individual's susceptibility, and/or the genetic analysis of SP-A2 gene in a population.

### FIELD OF THE INVENTION

The present invention relates to a method of detection of SP-A2 gene variants useful for prediction of predisposition to aspergillosis. The invention also provides primer and probe sequences useful in detecting these polymorphic variations in SP-A2 gene and their use in diagnosis and prediction of an individual's susceptibility to Allergic bronchopulmonary aspergillosis (ABPA). The invention is useful in molecular diagnosis, prediction of an individual's disease susceptibility and genetic analysis of SP-A2 gene in a population.

### BACKGROUND & PRIOR ART

About the Disease

Aspergillosis is a group of fungal diseases which include allergic bronchopulmonary aspergillosis (ABPA), aspergilloma, chronic necrotising aspergillosis, hypersensitivity pneumonitis and invasive aspergillosis. Aspergillus fumigatus, along with other less frequently reported species of Aspergillus such as A. flavus and A. niger, is the major causative fungus. Majority of the fungal allergies are due to Aspergillus species, with spores (conidia) and mycelia of the fungus as the infectious forms. A. fumigatus is an ubiquitous microbe and universal in distribution. Aspergillus species grow at temperatures of 15 to 53° C. in contrast to many fungi that do not grow above 35° C. Two major A. fumigatus induced diseases are an allergic form, Allergic bronchopulmonary aspergillosis (ABPA) and an invasive form, Invasive pulmonary aspergillosis. Pulmonary aspergillosis is a serious threat to those immunocompromised as a result of disease or therapy, and has been identified as a major cause of morbidity and mortality in asthmatic and cystic fibrosis patients [Daly et al, 2001].

Allergic Bronchopulmonary Aspergillosis (ABPA)

The allergic form of the Aspergillus induced disease is named as Allergic bronchopulmonary aspergillosis (ABPA), which is an immunological disease and depicts the immune mechanisms similar to that of asthma. ABPA is often encountered in patients of bronchial asthma (15%, 16 out of 107), cystic fibrosis (7.8%, 191 out of 12,447), sinusitis (13%, 3 out of 23), rhinitis (5%, 3 out of 62). [Chetty et al, 1985; Mastella et al, 2000; Panchal et al, 1997; Grammer et al, 1986]. A study with 35 patients of ABPA showed that 12 (33%) of them were misdiagnosed as patients of pulmonary tuberculosis and were treated with various antitubercular drugs [Behera et al, 1994]. A conglomeration of intertwined Aspergillus hyphae matted together with fibrin, mucus and cellular debris, within a pulmonary cavity or ectatic bronchus, is termed as Aspergilloma. Patients of aspergilloma usually have an underlying pulmonary disease such as fibrocystic sarcoidosis, cavitary tuberculosis or histoplasmosis, bullous emphysema, or fibrotic lung disease.

Diagnosis

Aspergillosis of the lung does not show characteristic clinico-radiological features to permit the diagnosis and should be considered in the differential diagnosis of tuberculosis, pneumonia, bronchiectasis, lung abscess and bronchial asthma. Early diagnosis of ABPA is important for the following reasons:

Firstly, to prevent irreversible damage of the bronchi and the lungs. Bronchiectasis and bronchiolitis are known sequelae of the disease and if undiagnosed in early stages, may lead to pulmonary fibrosis and respiratory failure. ABPA may be the cause of recurrent pneumonias in children and may increase the severity of asthma in some patients [Chetty et al, 1985].

Association of ABPA in cystic fibrosis may worsen the course and prognosis [Mastella et al, 2000].

ABPA has also been found in multiple members of a family and a need has been felt for screening all the family members of a newly diagnosed case.

Occurrence of ABPA is also known to be associated with pulmonary tuberculosis [Behera et al, 1994]. It has been observed that both diseases show similar clinical symptoms that cause a diagnostic dilemma.

Disease Loci Identified till now and Their Associations.

Infectious conidia of Aspergillus fumigatus are prevalent in the air and the population is exposed to them. However, occurrence of ABPA is limited to individuals with asthma, cystic fibrosis, atopic and other immunocompetent individuals. ABPA is a disease with immunological complexity. Genotype analysis of the T-cell clones (specific for the Asp f1 antigen), isolated from ABPA patients, showed that most of them are restricted by HLA-DR molecules (90% of the ABPA patients showing a phenotype either HLA-DR2, HLA-DR5 or both) [Knutsen et al, 1994; Chauhan et al, 1994; Chauhan et al, 1996; Chauhan et al, 1997]. HLA-DR molecules DR2, DR5, and possibly DR4 or DR7 contribute to susceptibility while HLA-DQ2 contributes to resistance. Further, a combination of these genetic elements may determine the outcome of ABPA in patients with cystic fibrosis and asthma. Detailed genotype analysis of ABPA patients revealed that susceptibility to ABPA is also associated with alleles of HLA-DR2 and HLA-DR5. The presence of DR4 or DR7 alleles in non-DR2/5 patients with ABPA suggested that these alleles may be contributing factors to pathogenesis. Chauhan et al, 2000, reported a significantly high frequency of HLA-DQ2 in patients without ABPA (67.4%), compared with patients with ABPA (20.5%) and normal control subjects (37.7%), suggesting that these alleles may confer protection in the population without ABPA.

Mutations in cystic fibrosis transmembrane protein (CFTR) encoding gene of cystic fiborsis patients lead to defective synthesis and regulation of cystic fibrosis transmembrane protein [Davis et al, 1996]. This protein is directly involved in the transportation of chloride ions. Such a defect in chloride transportation results in thick mucus secretion in these patients facilitating colonisation of pulmonary tract by other microbes. Presence of mutations in CFTR gene in ABPA patients suggest that there is some association of ABPA with cystic fibrosis [Miller et al, 1996]. In a recent study, the frequency of CFTR mutation carriers was observed to be significantly higher in ABPA patients (6 of 21 patients; 28.5%) than in control asthmatic subjects (2 of 43 subjects; 4.6%; p=0.01) [Marchand et al, 2001]. Hence, susceptibility and resistance to ABPA may be associated with certain genetic factors.

Role of Human Lung Surfactant Proteins in Aspergillosis:

Pulmonary surfactant proteins, SP-A and SP-D, are immune molecules which can directly interact with pathogens and allergens, stimulate immune cells and manipulate cytokine and chemokine profiles during host's immune response. Therapeutic administration of SP-A in murine model of invasive pulmonary aspergillosis can rescue mice from death. Treating mice, having ABPA, can suppress IgE levels, eosinophilia, pulmonary cellular infiltration and cause a marked shift from a pathogenic Th2 to a protective Th1 cytokine profile. These results highlight the potential of SP-A as novel therapeutics for lung allergy and infection. Therefore, the SP-A locus make particularly good candidate to be screened for predisposition to pulmonary infectious disease.

The human SP-A gene locus consists of 2 highly homologous functional genes, SP-Al and SP-A2, and a pseudogene located on 10q. Both functional genes consist of 4 coding exons. Several alleles that differ by a single amino acid had been identified in each SP-A gene (Floros et al., 1996). The alleles of the SP-A1 gene are denoted as ‘6A(n),’ and those of the SP-A2 gene as ‘1A(n)’ (Floros and Hoover, 1998). In Finland, Ramet et al. (2000) found that certain SP-A1 alleles, 6A(2) and 6A(3), and a SP-Al/SP-A2 haplotype, 6A(2)/1A(0), were associated with respiratory distress syndrome (RDS; 267450). The 6A(2) allele was overrepresented and the 6A(3) allele was underrepresented in infants with RDS. These associations were particularly strong among small premature infants born at gestational age less than 32 weeks. Ramet et al. (2001) reported that the frequency of specific surfactant protein-A haplotypes and genotypes differs between children with recurrent otitis media compared with a control population in Finland.

The prior art is lacking in any method that associates the allelic variants of SP-A2 gene to the ABPA susceptibility. The prior art is also lacking in any study that correlates the substructure of SP-A2 with predisposition to the ABPA.

Elevated levels of A. fumigatus specific IgE and IgG antibodies detectable by various serodiagnostic techniques is one of the important diagnostic criteria for ABPA. However, unfamiliarity with the diagnostic tests and nonavailability of certain serologic tests at clinical laboratories compounds the difficulty for the clinician. Reference standards of Aspergillus antigens for immunodiagnosis are not available till today either with the world health organisation (WHO) or any international agencies. This is mainly due to the complex nature of antigens of Aspergillus species, which require multiple purification processes.

This is the first demonstration that relates to the application of SNP's in human SP-A2 gene for use such as molecular diagnosis and prediction of an individual's disease susceptibility to ABPA or otherwise, and/or the genetic analysis of SP-A2 gene in Indian population. The novelty of the present invention is in providing a method for detecting and associating allelic variants of SP-A2 gene with the disease for prediction of an individual's predisposition to ABPA. [Daly P Kavanagh K 2001 Pulmonary aspergillosis: clinical presentation, diagnosis and therapy; Br. J. Biomed. Sci. 58 197-205. Chetty A Bhargava S Jain R K 1985 Allergic bronchopulmonary aspergillosis in Indian children with bronchial asthma; Ann. Allergy 54 46-49.; Mastella G Rainisio M Harms H K Hodson M E Koch C Navarro J Strandvik B McKenzie S G 2000 Allergic bronchopulmonary aspergillosis in cystic fibrosis. A European epidemiological study. Epidemiologic Registry of Cystic Fibrosis; Eur. Respir. J. 16 464-471. Panchal N Bhagat R Pant C Shah A 1997 Allergic bronchopulmonary aspergillosis: the spectrum of computed tomography appearances; Respir. Med. 91 213-219. Grammer L C Greenberger P A Patterson R 1986 Allergic bronchopulmonary aspergillosis in asthmatic patients presenting with allergic rhinitis; Int. Arch. Allergy Appl. Immunol. 79, 246-8.; Behera D Guleria R Jindal S K Chakrabarti A Panigrahi D 1994 Allergic bronchopulmonary aspergillosis: a retrospective study of 35 cases; Indian J. Chest Dis. Allied Sci. 36 173-179; Davis P B Drumm M Konstan M W 1996 Cystic fibrosis; Am. J. Respir. Crit. Care. Med. 154 1229-1256.]

### OBJECTS OF THE INVENTION

The main object of the present invention is to provide method of detection of allelic variants of human SP-A2 gene useful for prediction of predisposition to aspergillosis.

Another object is to provide allele specific primers and probes useful for detection of allelic variants of human SP-A2 gene.

Yet another object of the invention is to provide a method for establishing association of SP-A2 allelic variants with disease susceptibility.

Still another object of the invention is to provide a method for screening individuals carrying SP-A2 alleles predisposed to allergic bronchopulmonary aspergillosis.

### SUMMARY OF THE INVENTION

The present invention relates to allelic variants of human SP-A2 gene and provides specific primers suitable for detecting these allelic variants for applications such as molecular diagnosis and prediction of an individual's disease susceptibility to ABPA or otherwise, and/or the genetic analysis of SP-A2 gene in Indian population. Two major A. fumigatus induced diseases are in the allergic form, Allergic bronchopulmonary aspergillosis (ABPA) and an invasive form, Invasive pulmonary aspergillosis. Pulmonary aspergillosis is a serious threat to those immunocompromised as a result of disease or therapy, and has been identified as a major cause of morbidity and mortality in asthmatic and cystic fibrosis patients. Pulmonary surfactant protein, SP-A directly interacts with pathogens and allergens, stimulates immune cells and manipulates cytokine and chemokine profiles during host's immune response.

Administration of SP-A to mice, having ABPA, can suppress IgE levels, eosinophilia, pulmonary cellular infiltration and cause a marked shift from a pathogenic Th2 to a protective Th1 cytokine profile. These results highlight the potential of SP-A as novel therapeutics for lung allergy and infection. The human SP-A gene locus consists of 2 highly homologous functional genes, SP-A1 and SP-A2, and a pseudogene located on 10q. The novelty of the present invention is in providing a method for detecting and associating allelic variants of SP-A2 gene with the disease for prediction of an individual's predisposition to ABPA.

### DETAILED DESCRIPTION OF THE INVENTION

The present invention relates to the detection of allelic variants of the human SP-A2 gene and their utility in predicting an individual susceptibility to the ABPA.

The region containing the SNP's was PCR amplified using the primers SP-A2 F and SP-A2 R. Approximately 100 ng of genomic DNA was amplified in a 50 μl reaction volume containing a final concentration of 5 mM Tris, 25 mM KCl, 0.75 mM magnesium chloride (MgCl2), 0.05% gelatin, 20 pM of each primer and 1.5 U of Taq DNA polymerase. Samples were denatured at 95° C. for 5 min followed by 30 cycles of denaturation (95° C. for 1 min), annealing (70° C., 1 min), extension (72° C., 1 min) and a final extension of 7 min at 72° C. in a Perkin Elmer Gene Amp PCR System 9600. The PCR product was purified from band cut out of the agarose gel using QIA Quick gel extraction kit (QIAGEN) and was directly sequenced using dye terminator chemistry on an ABI Prism 377 automated DNA sequences with the PCR primers.

Accordingly, the present invention provides method for detection of human SP-A2 gene variants, useful for prediction of predisposition to aspergillosis, said method comprising the steps of:

1. Designing and synthesising specific oligonucleotide primers for PCR amplification of exon 2 of human SP-A2.

2. Amplifying genomic DNA of ABPA patients and normal control individuals using the above said primers.

3. Sequencing the amplified PCR product and identifying sequence variation computationally by comparing it with the already reported sequence of human SP-A2 gene (accession No. M30838)

4. Screening Normal Control individuals and ABPA patients for novel single nucleotide polymorphisms by sequencing amplified exon 2 of SP-A2 gene.

5. Computing the frequencies of G/C alleles (SNP at position 1629) and A/G alleles (SNP at position 1640) in normals and ABPA patients.

6. Establishing the association of G/C and A/G alleles with the ABPA disease based on their frequencies distribution on normal and ABPA patients.

Predicting the resistance or susceptibility to the ABPA based on the nucleotide present at the polymorphic sites in the individual tested, wherein C allele (at nucleotide position 1629) and A allele (at nucleotide position 1640) being at low risk and G allele (at nucleotide position 1629) and G allele (at nucleotide position 1640) at high risk to the disease.

In an embodiment, the primers suitable for amplification of SP-A2 gene region containing one or more polymorphic sites, all selected from the group consisting of SEQ ID No:1, SEQ ID No:2, SEQ ID No. 3, SEQ ID No. 4 & compliments thereof.

In still another embodiment, allelic variants of SP-A2 gene have C/G and A/G haplotypes,

Further, the invention provide a diagnostic kit for the detection of SNP haplotypes C/G or A/G comprising suitable primers and probes selected from polynucleotide sequences under SEQ ID No. 1-4

In another embodiment of the invention a nucleic acid vector may contain the allelic variants of SP-A2 gene.

In an embodiment of the invention, primer suitable for amplification of SP-A2 gene region containing one or more polymorphic sites are provided, said primers selected from the group comprising.

(a) 5′CTG CGT GCG AAG TGA AGG ACG TTT GTG TTG 3′
(Forward)
(b) 5′GAC CCC CAT CAC CCC TGT GTA ACT GAC TTC 3′
(Reverse)
(c) 5′TGC CTG GAG CCC CTG GTG TCC CTG GAG AGC 3′
(Forward)
(d) 5′TGC CTC GTC CGC ATT CAC CCT TCA GAC TGC 3′
(Reverse)

The allelic variants of human SP-A2 gene may comprise one or more of the following SNP's as compared with the human SP-A2 complete cDNA sequence in the database (GenBank Acc NO. M 30838)

TABLE I

| | Site of change | Base change | Amino acid alteration |
|---|---|---|---|
| A | Nucleotide position | C-G | Proline-Alanine |
| | 1629 | | |
| B | Nucleotide position | A-G | Arginine-Arginine |
| | 1640 | | |

The site of change is in accordance with the human SP-A2 complete cDNA sequence in the database (GenBank Acc No. M 30838).

The invention also provides a method of analysing a nucleic acid from an individual for the presence of base at anyone of the polymorphic site shown in Table-I. This type of analysis can be performed on a plurality of individuals who are tested either for the presence or for predisposition to ABPA. The susceptibility to the disease can then be established based depending on the base or set of basis present at the polymorphic sites in the individuals tested.

Invention also provides oligonucleotide sequences (as listed in SEQ ID NO. 1-4), suitable for use as allele specific primers and probes for the detection of polymorphic sites listed in table-I.

Further, a diagnostic kit comprising one or more of the allele specific primers or probes along with the required buffer and accessories suitable for identification of SP-A2 allelic variants to establish an individual's susceptibility to ABPA is also included in the invention.

Eukaryotic expression vectors comprising a DNA sequence coding for a protein or a peptide according to the invention are new materials and also included in the invention. Host cells, for example cloned human cell lines, can be transformed using the new expression vectors and are also included in the invention.

### BRIEF DESCRIPTION OF THE DRAWINGS

In the drawing (s), accompanying the specifications.

FIG. 1 is a schematic representation of the two SNP's in SP-A 2 gene. The top line depicts the position of the four exons of the SP-A2 gene. The second line shows the relative locations of the two polymorphic sites. Both the polymorphisms are also shown in the sequence content of the gene.

The manner in which the above mentioned features, advantages and objects of the invention as well as others which will become clear are attained and can be understood in detail by the particular description of the invention and the examples which are provided to illustrate preferred embodiments of the invention and are not be considered limiting in their scope.

Other and further aspects, features, and advantages of the present invention will be apparent from the following description of the preferred embodiments of the invention given for the purpose of disclosure. Alternative embodiments of the invention can be envisaged by those skilled in the art. All such alternative embodiments are intended to lie within the scope of this invention of this invention.

Polymorphism of the Invention

As a first step to the present invention, the applicants carried out the PCR amplification of exon 2 of human SP-A2 complete cDNA sequence submitted by White R. T. et.al. in the database (GenBank Acc No. M 30838). The sequencing of the purified PCR product revealed two SNP's in the exon 2 of human SP-A2 gene.

The present invention provides a sequence for the allelic variants of human SP-A2 gene comprising one or more of the following SNP's compared with the human SP-A2 complete cDNA sequence in the database.

TABLE I

| | Site of change | Base change | Amino acid alteration |
|---|---|---|---|
| A | Nucleotide position | C-G | Proline-Alanine |
| | 1629 | | |
| B | Nucleotide position | A-G | Arginine-Arginine |
| | 1640 | | |

The site of change is in accordance with the human SP-A2 complete cDNA sequence in the database (GenBank Acc No. M 30838).

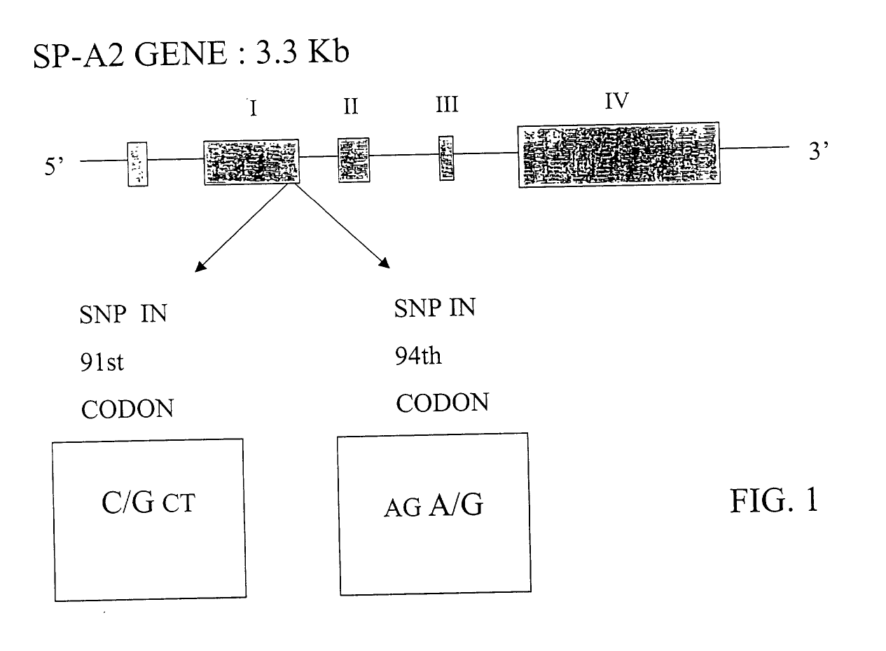

The first polymorphic site (A) as shown in FIG. 1, had either a C or a G. The second polymorphic site (B) contains either A or a G base. While the first substitution changes the amino acid sequence from Proline to Alanine, the second substitution is neutral. For example, the nucleotide sequence of the allelic variant of exon 2 of human SP-A2 gene having polymorphic sites as listed in table-I may be

5′ gccccatggg tccacctgga gaaatgccat gtcctcctgg aaatgatggg ctgcctggag cccctggtat ccctggagag tgtggagaga agggggagcc tggcgagagg ggccctccag 3′

In the above sequence the SNP's (A) and (B) are at nucleotide position 1629 and 1640 respectively and are shown in bold.

II Association Analysis with the Disease.

Analysis of these two SNP's in the 150 Normal and 40 ABPA patient chromosomes revealed that two haplotypes, possible with each SNP in a biallelic polymorphic system, were observed. The frequency in Normal and ABPA patient chromosome is summarised in Table-II.

TABLE II

| | No. of | | | | |
|---|---|---|---|---|---|
| | chromosomes | SNP at 1629 position | | SNP at 1640 position | |
| | (N) studied | % CCT allele | % GCT allele | % AGA allele | % AGG allele |
| Normal | 150 | 75.33 | 24.67 | 95.33 | 4.67 |
| ABPA | 40 | 65.0 | 35.0 | 70.0 | 30.0 |
| patient | | | | | |

Further, studies on ABPA patient chromosomes revealed a high significant difference in the distribution of the two SNP's in the Normal and the ABPA patient chromosome (Table-III).

TABLE III

| | SNP (G vs C) at 1629 | SNP (G vs A) at 1640 |
|---|---|---|
| | position | position |
| ODDS RATIO (ABPA | 1.64 | 8.76 |
| patient vs Normal) | | |
| Chi-square | 1.717 | 22.519 |
| p-value | 0.1900 | 0.0000 |

III Diagnostic Kit

The invention further provides diagnostic kit, comprising at least one or more allele specific oligonucleotides as described in SEQ ID 1-4.

Often the kit contain one or more pair of allele specific oligonucleotides two different forms of polymorphisms. In some kits, the allele specific oligonucleotide are provided immobilised to a surface for example, the same substrate can comprise allele specific oligonucleotide probes for detecting at least one or all of the polymorphism shown in Table-I. Optional additional components of the kit includes, for example, restriction enzymes, reverse transcriptase or polymerase, the substrate nucleoside triphosphate, means use to label (for example, an evident enzyme conjugate and enzyme substrate and chromogen if the label is biotin) and the appropriate buffer for the reaction, PCR, or hybridisation reaction. Usually, the kit also contains instructions for carrying out the methods.

Nucleic Acid Vectors:

Variant genes can be expressed in an expression vector in which a variant gene is operably linked to a native or other promoter. Usually the promoter is a eukaryotic promoter for expression in mammalian cell. The transcription regulation sequence typically include a heterologous promoter and optionally an enhancer which is recognised by the test. The structure of an appropriate promoter, for example, Trp, Lac, phage promoter, glycolytic enzyme promoters and tRNA promoter depends on the host selected. Commercially available expression vectors can also be used. Suitable host cells include bacteria such as E. coli, Yeast, filamentous fungi, insect cells, mammalian cells, typically immortalised example, mouse, CHO, human and monkey cell lines and derivatives thereof. Preferred host cells are able to process the variant gene product to produce an appropriate mature polypeptide.

The invention further provides transgenic, non-human animals, capable of expressing an endogenous variant gene and/or having one or both allele of an endogenous variant gene inactivated. Expression of an endogenous variant gene is usually achieved by operably linking the gene to a promoter and optionally an enhancer, and microinjecting the construct into a zygote. Inactivation of endogenous variant genes can be achieved by forming a transgene in which a cloned variant gene is then introduced into an embryonic stem cell, where it undergoes homologous recombination with an endogenic variant gene. Mice and other rodents are preferred animals. Such animals provide useful drug delivery systems.

### EXAMPLE 1

Identification of Allelic Variants of SP-A2 Gene

This example describes the identification of allelic variant of human surfactant protein A 2 gene by PCR and sequencing using certain oligonucleotide primers. According to the invention DNA was extracted from human peripheral blood leukocytes using a modification of salting out procedure. The concentration of the DNA was determined by measuring the optical density of the sample, at a wavelength of 260 nm. The DNA was then amplified by PCR by using the oligonucleotide primers.

5′CTG CGT GCG AAG TGA AGG ACG TTT GTG TTG 3′
(Forward)
5′GAC CCC CAT CAC CCC TGT GTA ACT GAC TTC 3′
(Reverse)
5′TGC CTG GAG CCC CTG GTG TCC CTG GAG AGC 3′
(Forward)
5′TGC CTC GTC CGC ATT CAC CCT TCA GAC TGC 3′
(Reverse

The sample were denatured at 950 C for 5 minutes followed by 28 cycles of denaturation (950 C, 1 minutes), annealing (700 C, 1 minute), extension (720 C, 1 minute) and a final extension of 7 minutes at 720 C in a PE GeneAmp PCR System 9600. This reaction produced a DNA fragment of 459 bp when analysed by Genescan analysis by using ABI Prism 377 automated DNA sequencer. The PCR product was purified from band cut out of agarose gel using a Qiaquick gel extraction kit (Qiagen) and both the strands of the PCR product were directly sequenced using gel terminator chemistry on an ABI Prism 377 automated DNA sequencer with PCR prisms. The PCT products were shown to be identical to the human SP-A2 mRNA, complete CDS sequence in the databse (acc. no. M30838), submitted by White R. T. et al except for the previously mentioned 2 single base changes as listed in table 1.

### EXAMPLE 2

Nucleotide Sequence of the Allelic Variant of SP-A2 Gene.

The nucleotide seq. of the allelic variant of SP-A 2 gene derived using the method as described in example 1.

In the above sequence the 2 SNP's as given in table 1 are at nucleotide position 1629 and 1640.

### EXAMPLE 3

Patients with A Allele at 1640 Position are at Nearly Zero Risk for the ABPA Disease.

A method as described in example 1 is applied to a series of DNA samples extracted from ABPA positive individuals and normal controls. There is observed a statistically significant difference (At position 1629 p=0.1900 and at position 1640 p=0.0000) in the frequency distributions of the SNP haplotypes generated using SNP in normal and ABPA patient SP-A2 chromosome. The results obtained are summarized in table below

TABLE III

| | SNP (G vs C) at 1629 | SNP (G vs A) at 1640 |
|---|---|---|
| | position | position |
| ODDS RATIO (ABPA | 1.64 | 8.76 |
| patient vs Normal) | | |
| Chi-square | 1.717 | 22.519 |
| p-value | 0.1900 | 0.0000 |

A strong association of G (at 1629 position) and G (at 1640 position) haplotypes with ABPA disease chromosome indicated that SP-A2 alleles with the G (at 1629 position) and G (at 1640 position) haplotypes are predisposed to the disease. Therefore, these SNP haplotypes in the human SP-A2 gene could be used as a method of establishing individual risk to ABPA. The association of G (at 1629 position) and G (at 1640 position) haplotypes with the ABPA disease was studied in Indian population. However, C (at 1629 position) and A (at 1640 position) haplotypes being at low risk and G (at 1629 position) and G (at 1640 position) haplotypes being at high risk for ABPA disease, can be expected to hold true for other human population also.

### EXAMPLE 4

Nucleic Acid Vector Containing the SP-A2 Variant Sequences.

Expression vectors and host cells transformed with allelic variants of the SP-A2 gene, containing one or more polymorphic sites as listed in table 1 can be proposed, for example as detailed below.

Allelic variant of SP-A2 gene can be expressed in an expression vector in which the variant gene is operably linked to a native or other promoter. Usually the promoter is a eukaryotic promoter for expression in mammalian cell. The transcription regulation sequence typically includes a heterologous promoter and optionally an enhancer which is recognised by the test. The structure of an appropriate promoter, for example, Trp, Lac, phage promoter, glycolytic enzyme promoters and tRNA promoter depends on the host selected. Commercially available expression vectors can also be used.

The means of introducing the expression construct into a host cell will depend on particular construct and the target host. Suitable means include fusion, conjugation, transfection, transduction, electroporation or injection. A wide variety of host cells can be employed for expression of the variant gene, both prokaryotic and eukaryotic. Suitable host cells include bacteria such as E. coli, Yeast, filamentous fungi, insect cells, mammalian cells, typically immortalised example, mouse, CHO, human and monkey cell lines and derivatives thereof. Preferred host cells are able to process the variant gene product to produce an appropriate mature polypeptide.

Advantages:

The invention shall be useful to establish genotype or base variation of SP-A2 gene. The information may be useful for molecular diagnosis, prediction of an individual's disease susceptibility to ABPA, prognosis and/or the genetic analysis of ABPA gene in a population. The frequency of these variants can also be used to predict the prevalence of ABPA disease among various populations.

1. A method of detection of human SP-A2 gene variants useful for prediction of predispositon to aspergillosis, said method comprises:
(a) designing and synthesizing oligonucleotide primers for PCR amplification of Exon II region of human SP-A2 gene,
(b) amplifying genomic DNA of ABPA patients and normal control individuals using the said primers of step (a),
(c) sequencing the amplified PCR product and identify the sequence variations computationally by comparing it with the already existing sequence of human SP-A2 gene,
(d) screening normal control individuals and ABPA patients' single nucleotide polymorphisms by sequencing of the amplified region of the individuals using the said primers of step (a),
(e) computing the frequency of C/G haplotypes at 1629 position and A/G haplotypes at 1640 position,
(f) establishing the association of G (at 1629 position) and G (at 1640 position) haplotypes with the ABPA disease based on their frequency distribution in normals and ABPA patients, wherein presence of C at 1629 position and A at 1640 position in the haplotypes is indicative of the individual being at low risk to aspergillosis and presence of G at 1629 position and G at 1640 position in the haplotype is indicative if the individual being at high risk to aspergillosis.

2. A method as claimed in claim 1 wherein the primers suitable for amplification of SP-A2 region containing 1 or more polymorphic sites are selected from the group
(a) 5′CTG CGT GCG AAG TGA AGG ACG TTT GTG TTG 3′
(Forward)
(b) 5′GAC CCC CAT CAC CCC TGT GTA ACT GAC TTC 3′
(Reverse)
(c) 5′TGC CTG GAG CCC CTG GTG TCC CTG GAG AGC 3′
(Forward)
(d) 5′TGC CTC GTC CGC ATT CAC CCT TCA GAC TGC 3′
(Reverse)

3. A method as claimed in claim 1 wherein, the length of oligonucleotide primers is in the range 5-100 bases.

4. A method as claimed in claim 1 wherein allelic variants of SP-A2 gene have C/G and A/G haplotypes.

5. A diagnostic kit for the detection of Single nucleotide polymorphism haplotypes (C/G at 1629 position and A/G at 1640 position) comprising primers selected from the SEQ ID No. 1-4.

6. Primer suitable for amplification of SP-A2 gene region containing one or more polymorphic sites, said primer selected from the group consisting of sequence given under SEQ ID No. 1-4 and compliments thereof.


SEQUENCE LISTING (5 sequences)

SEQ ID NO: 1 - DNA, 30 bp, Homo sapiens
ctgcgtgcgaagtgaaggacgtttgtgttg

SEQ ID NO: 2 - DNA, 30 bp, Homo sapiens
gacccccatcacccctgtgtaactgacttc

SEQ ID NO: 3 - DNA, 30 bp, Homo sapiens
tgcctggagcccctggtgtccctggagagc

SEQ ID NO: 4 - DNA, 30 bp, Homo sapiens
tgcctcgtccgcattcacccttcagactgc

SEQ ID NO: 5 - DNA, 120 bp, Homo sapiens
gccccatgggtccacctggagaaatgccatgtcctcctggaaatgatgggctgcctggagcccctggtatccctggagagtgtggagagaagggggagcctggcgagaggggccctccag